(19) United States
(12) Patent Application Publication
(10) Pub. No.: US 2023/0001028 A1
(43) Pub. Date: Jan. 5, 2023

(54) INFRARED DRYING SYSTEMS

(71) Applicant: BluTEQ, LLC, Eden Prairie, MN (US)

(72) Inventors: Christopher P. Moarn, Edina, MN (US); Paul D. Rudd, Minneapolis, MN (US)

(21) Appl. No.: 17/528,961

(22) Filed: Nov. 17, 2021

Related U.S. Application Data
(63) Continuation of application No. 16/100,089, filed on Aug. 9, 2018, now abandoned.
(60) Provisional application No. 62/543,075, filed on Aug. 9, 2017.
(60) Provisional application No. 62/542,876, filed on Aug. 9, 2017.

(51) Int. Cl. A61L 2/08; F26B 3/30; F26B 17/20; A61L 2/04; A61L 2/26; F26B 11/06; F26B 11/04

(52) U.S. Cl. CPC A61L 2/085; F26B 3/30; F26B 17/20; A61L 2/04; A61L 2/26; F26B 11/0486; F26B 11/045; A01K 1/0135

(57) ABSTRACT

An infrared heating system is disclosed that includes an enclosed tube extending from a first open end to a second open end, the enclosed tube having a sidewall opening in a sidewall of the tube at a location between the first and second open ends; an infrared heater mounted to the exterior of the enclosed tube such that heating elements of the infrared heater are exposed to the tube sidewall opening; and an auger disposed within the enclosed tube, the auger being driven by a drive system such that material fed into the enclosed tube at the first open end is transported to the second open end of the tube and is exposed to the heating elements of the infrared heater as the material is transported between the first and second open ends.

### RELATED APPLICATIONS

This application is a continuation of U.S. application Ser. No. 16/100,089, filed Aug. 9, 2018, now abandoned; which claims priority to U.S. Provisional Application Ser. No. 62/542,876, filed on Aug. 9, 2017 and U.S. Provisional Patent Application Ser. No. 62/543,075, filed on Aug. 9, 2017, the entireties of which are incorporated by reference herein.

### SUMMARY

This disclosure relates to systems for drying wet organic solids, for example biosolids, through the use of radiant heat generated from at least one infrared heating element. Wet organic solids must be dried in many applications, for example, solids from wastewater treatment systems. In many instances, wet organic solids are dried by convection only. This process is both time consuming and energy intensive. The utilization of radiant heat provides for the possibility of lower operating costs and shortened processing times.

At least some known drying systems use a rotatable cylindrical drum to receive the wet organic solids and process the wet solids into dry solids. In these drying systems the drum is continuously rotated to churn the wet solids and lift the solids residing at the bottom of the drum towards the top layer for a direct line with the radiant heat. However, the cylindrical drums are considered a complex shape that includes curved sheet pieces that are time intensive to form, thereby increasing the manufacturing costs of the drums and the overall drying system.

In one example process, an infrared drying system is used for drying of dairy cow bedding through the use of an infrared heating system and auger conveying system. The process begins with receiving cow manure from a dewatering/press machine. The product is delivered to the open end of the auger and the product is then conveyed through the length of the auger in a conveyor channel or tube. Infrared heaters placed on top of the auger will dry the product as it passes underneath the heaters. During this process, the cow manure is heated between 176 degrees F. and 280 degrees F. as it moves toward the opposite end of the auger. As the cow manure moves to the open exit after approximately 10 minutes in the auger, the new product should be pathogen free and approximately 20% drier. The new drier, sterilized bedding is now ready to be used as bedding for dairy cows.

In one example, an infrared treatment system for killing pathogens in a waste stream is disclosed. The system can include a conveyance tube extending from an inlet end to an outlet end, the conveyance tube having a double wall construction with insulation disposed between an inner wall and an outer wall, an infrared heater mounted to the conveyance tube such that heating elements of the infrared heater are exposed to an interior of the conveyance tube, and a first auger and a second auger are disposed within the enclosed tube. In some examples, the first and second augers are driven by a drive system in a counter-rotating configuration such that material fed into the enclosed tube inlet is transported to the outlet end of the tube, and is exposed to the heating elements of the infrared heater as the material is transported between the inlet and outlet open ends.

In some examples, the infrared heater includes a heating element operating at about 2,000 degrees F.

In some examples, the inner wall is formed from stainless steel.

In some examples, the treatment system is constructed from multiple modules, wherein at least two of the modules includes an infrared heater and a section of the first and second augers.

In some examples, the infrared treatment system modules each have a length of about 10 feet.

In some examples, the infrared heaters include ferritic iron-chromium-aluminum alloy wire heating elements.

In some examples, the system further includes a water injection system, the water injection system including a valve operable to inject water into the conveyance tube at a predetermined sensor temperature.

In some examples, the inner wall includes four straight segments separated by four bend lines.

In some examples, the system further includes a wedge structure extending between and parallel to the first and second augers.

In one example, a process for treating livestock manure is disclosed. The process can include the steps of receiving livestock manure at an inlet end of a conveyance tube; transporting the livestock manure to an outlet end of the conveyance tube with a pair of counter-rotating augers disposed within the enclosed tube; exposing the livestock manure to heating elements of an infrared heater while the livestock manure is being transported from the inlet open end to the outlet open end, wherein the heating elements are operating at a temperature of at least 2,000 degrees; and setting at least one of an auger speed and a heating element output such that the livestock manure is treated to eliminate 99% or more of the pathogen content of the livestock manure and to have moisture content of not less than 50% by weight.

In some examples, the process includes injecting water into the conveyance tube and deactivating the infrared heaters when a predetermined temperature threshold is exceeded within the conveyance tube.

In some examples, the livestock manure has an initial moisture content of about 65 to 70 percent by weight.

In some examples, the cow manure is heated between 176 degrees F. and 280 degrees F. as it moves toward the opposite end of the auger. As the cow manure moves to the open exit after approximately 10 to 12 minutes in the auger, the new product should be pathogen free and approximately 20% drier. The new drier, sterilized bedding is now ready to be used as bedding for dairy cows.

A variety of additional aspects will be set forth in the description that follows. The aspects can relate to individual features and to combinations of features. It is to be understood that both the forgoing general description and the following detailed description are exemplary and explanatory only and are not restrictive of the broad inventive concepts upon which the examples disclosed herein are based.

### BRIEF DESCRIPTION OF THE DRAWINGS

FIG. 1 is a perspective view of an infrared drying system having features that are examples of aspects in accordance with the principles of the present disclosure.

FIG. 2 is an interior perspective view of the infrared drying system shown in FIG. 1.

FIG. 3 is an interior side view of the infrared drying system shown in FIG. 1.

FIG. 4 is a longitudinal cross-sectional side view of the infrared drying system shown in FIG. 1.

FIG. 5 is a transverse cross-sectional side view of the infrared drying system shown in FIG. 4 taken along line 5-5.

FIG. 6 is an exploded perspective view of the infrared drying system shown in FIG. 1 including a drum that has features that are examples of aspects in accordance with the principles of the present disclosure.

FIG. 7 is a back perspective view of the drum shown in FIG. 6.

FIG. 8 is a side view of the drum shown in FIG. 6.

FIG. 9 is a front view of the drum shown in FIG. 6.

FIG. 10 is a back view of the drum shown in FIG. 6.

FIG. 11 is a longitudinal cross-sectional side view of the drum shown in FIG. 8 taken along line 11-11.

FIG. 12 is a longitudinal cross-sectional perspective view of the drum shown in FIG. 11.

FIG. 13 is a longitudinal cross-sectional bottom view of the drum shown in FIG. 8 taken along line 13-13.

FIG. 14 is a cross-sectional side view of an infrared drying system having features that are examples of aspects in accordance with the principles of the present disclosure.

FIG. 15 is a top view of the infrared drying system shown in FIG. 14.

FIG. 16 is exploded side view of the infrared drying system shown in FIG. 14.

FIG. 17 is a side view of a modular infrared drying system having features that are examples of aspects in accordance with the principles of the present disclosure, with some elements of the system being shown transparent such that internal components can be more easily viewed.

FIG. 18 is a side view of one of the modules of the modular infrared drying system shown in FIG. 17, with some elements of the system being shown transparent such that internal components can be more easily viewed.

FIG. 19 is a top view of the infrared drying system module shown in FIG. 18, with some elements of the system being shown transparent such that internal components can be more easily viewed.

FIG. 20 is an end view of the infrared drying system module shown in FIG. 18, with some elements of the system being shown transparent such that internal components can be more easily viewed.

FIG. 21 is a process diagram utilizing the drying system shown in FIG. 14.

### DETAILED DESCRIPTION

Reference will now be made in detail to the exemplary aspects of the present disclosure that are illustrated in the accompanying drawings. Wherever possible, the same reference numbers will be used throughout the drawings to refer to the same or like structure.

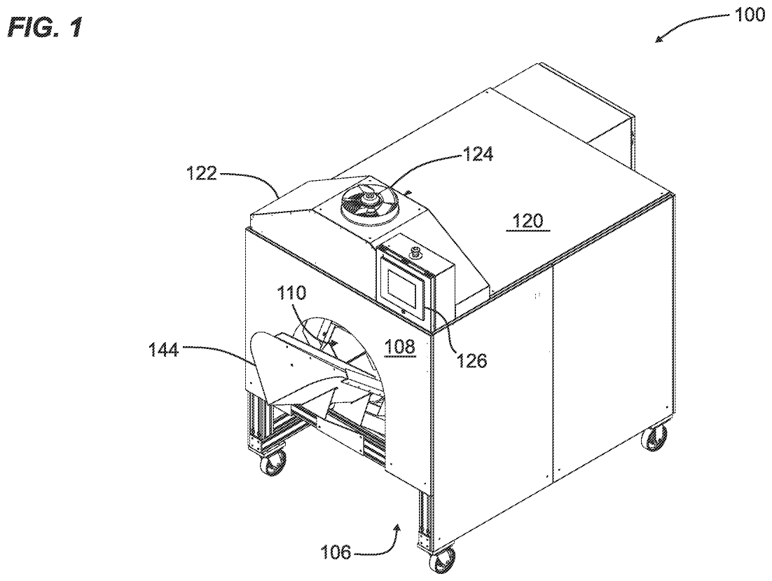

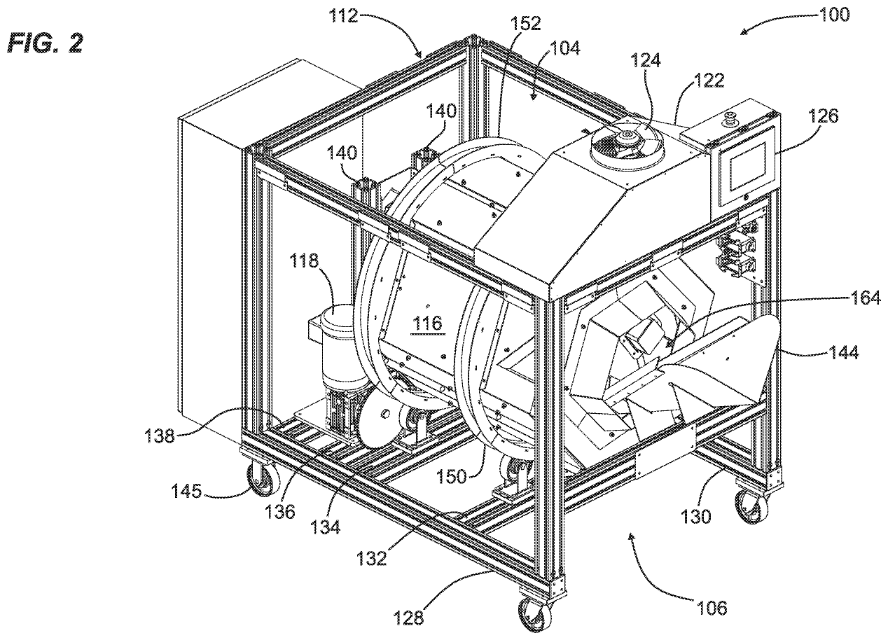

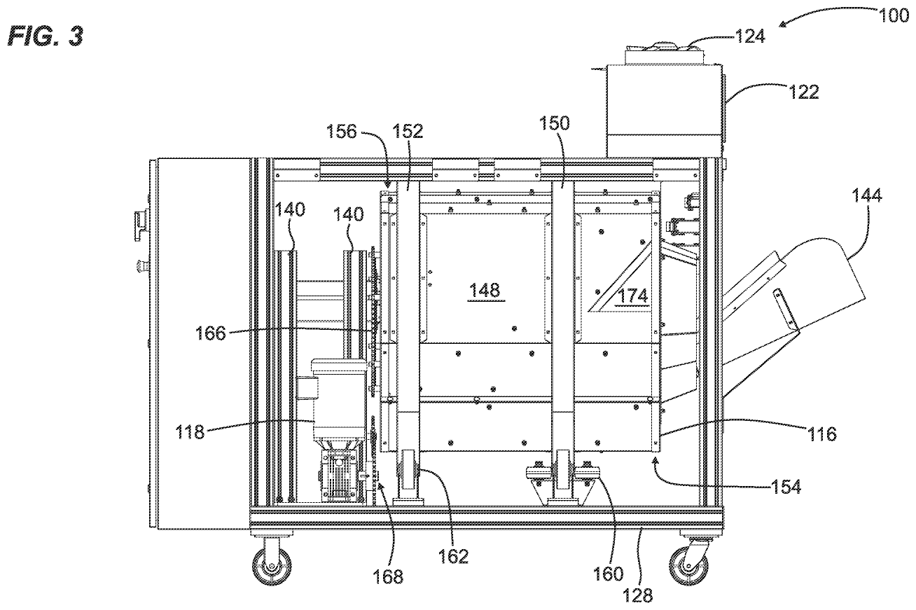

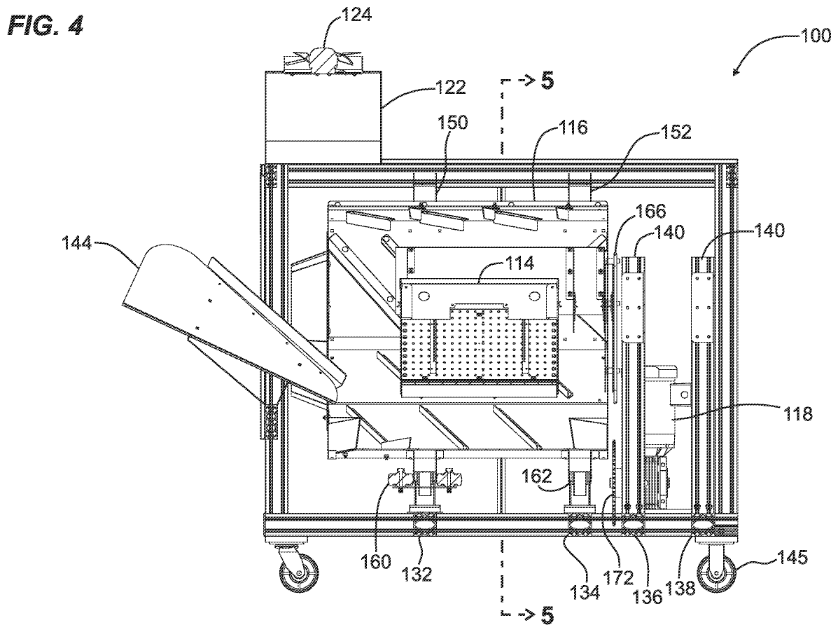

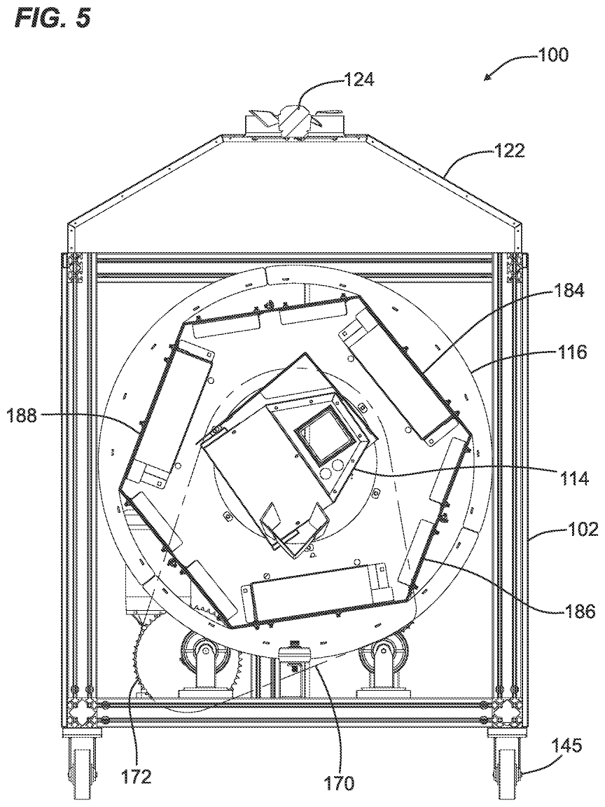

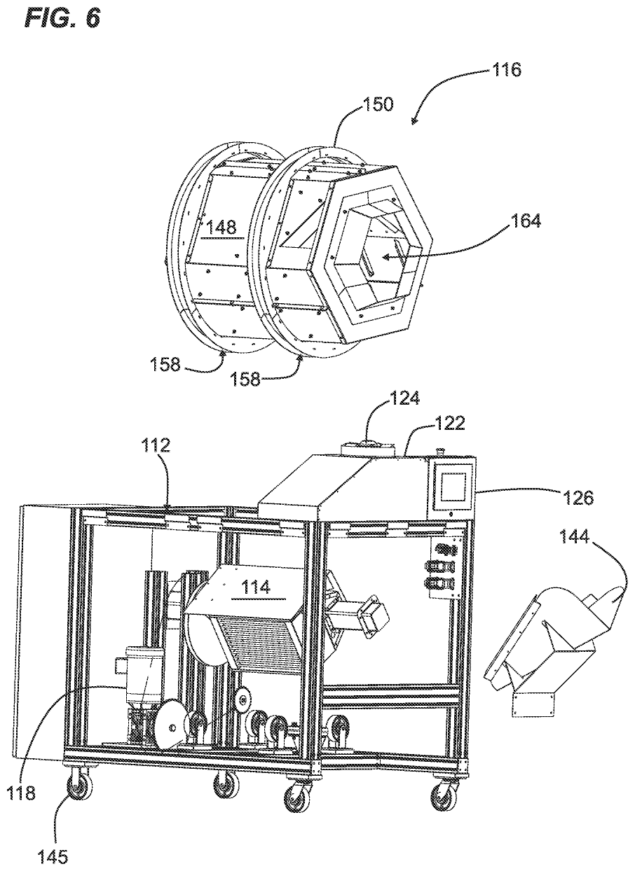

Referring to FIGS. 1-6, an infrared drying system 100 is disclosed. Infrared drying system 100 is used for lowering the moisture content of wet solids to result in dried solids. As most easily seen in FIGS. 1 and 2, infrared drying system 100 includes a housing 102 defining an interior space 104 and an open bottom 106. Housing 102 includes a front wall 108 defining a process opening 110 for enabling wet solids to enter into interior space 104 while the open bottom 106 is for enabling the dried solids to exit housing 102. In one example, infrared drying system 100 is about 6.2 feet long, about 4 feet wide, and about 5.6 feet tall. One will appreciate that other dimensions may be selected depending on production requirements.

Within housing 102, the infrared drying system 100 includes a frame assembly 112 supporting an infrared heating system 114 mounted within a rotatable drum 116. The drum 116 receives the wet organic solids, such as egg shells, for drying within infrared drying system 100. Drum 116 may be rotated by a drive motor 118 also mounted on frame assembly 112. Mounted on a roof 120 of housing 102, infrared drying system 100 includes an exit air system 122 in flow communication with interior space 104. Exit air system 122 includes an exit fan 124 having a blower (not shown) to channel steam out of interior space 104 and into the ambient air surrounding housing 102. Also mounted on the roof 120, infrared drying system 100 includes a control panel 126. Control panel 126 enables monitoring and control of all of the process variables relating to infrared drying system 100. For example, control panel 126 may include a color touch-screen display for controlling at least one of the infrared heating system 114, drive motor 118, and exit air system 122. The display enables viewing and entry of data pertinent to operation of infrared drying system 100 in both numerical and graphical form.

Frame assembly 112 includes two bottom rails 128, 130 having two cross-rails 132, 134 for supporting drum 116, and two cross-rails 136, 138 for supporting drive motor 118. Four vertical rails 140 extend into the interior space 104, two on each cross-rail 136, 138, for supporting infrared heating system 114 via at least one bracket. The frame assembly 112 also includes a front cross-rail 142, above the bottom rails 128, 130, for supporting a loading assembly 144. The loading assembly 144 enables the wet solids to be loaded into drum 116 from outside of housing 102. Frame assembly 112 may be of any construction suitable to support the weight of housing 102, and the associated components. For example, frame assembly 112 is constructed of welded tubular members having casters 145 coupled to the base such that infrared drying system 100 may be moveable.

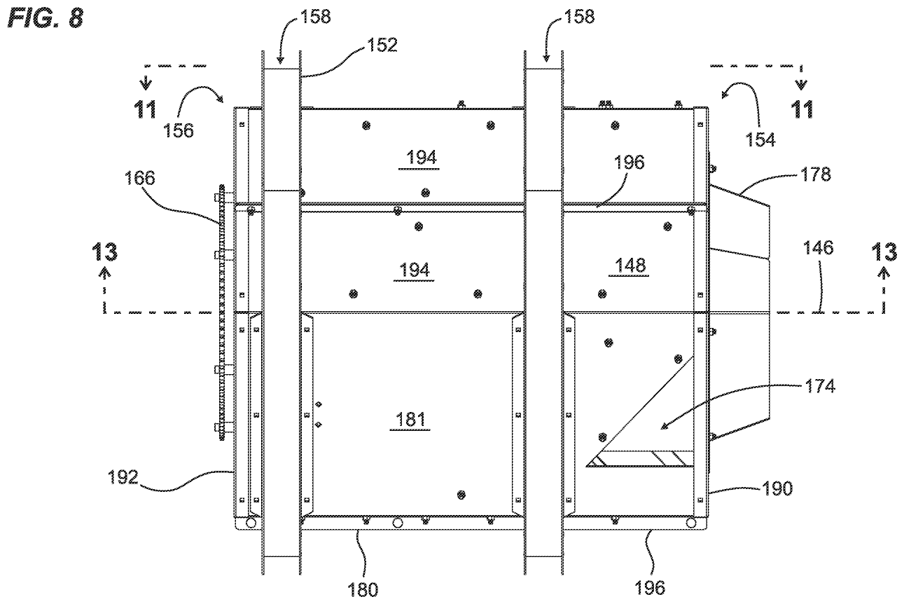

In the example, drum 116 extends along a longitudinal axis 146 (shown in FIG. 8) and is hexagonally-shaped in cross-section with an exterior surface 148. Extending from exterior surface 148 are two circumferential ribs 150, 152, one rib 150 is positioned towards a front end 154 of drum 116 and the other rib 152 is positioned towards a back end 156 of drum 116. Both ribs 150, 152 are cylindrical about the longitudinal axis 146 and define a radial U-shaped channel 158 that extends circumferentially around the perimeter of the rib 150, 152. Drum 116 is rotatably supported on cross-rails 132, 134 by roller assemblies 160, 162. As shown, two roller assemblies 160 are positioned on cross-rail 132 and include a plurality of casters that are received within channel 158 and outside of channel 158 to engage rib 150 such that drum 116 may rotate within housing 102 and is restricted from longitudinal movement. Two roller assemblies 162 are also positioned on cross-rail 134 and include a single caster that is received within channel 158 and engage rib 152 such that drum 116 may rotate within housing 102.

The front end 154 of drum 116 includes an opening 164 that enables the wet solids to be channeled into drum 116. The back end 156 of drum 116 includes a gear 166. Gear 166 can be coupled to drive motor 118 through a transmission system 168 such that drum 116 is rotatable about longitudinal axis 146 in both clockwise and counter-clockwise directions. As shown, transmission system 168 includes, for example, a drive belt 170 and a gear reducer 172. In alternative examples, drive motor 118 may be driven by a variable frequency drive and connected directly to drive belt 170 without reliance on gear reducer 172.

In operation, infrared drying system 100 enables wet solids to be processed into dried solids. More specifically, wet solids can be loaded into drum 116 through loading assembly 144. The drum 116 is rotated clockwise via drive motor 118 about the fixed infrared heating system 114 that is disposed therein to dry the wet solids. The infrared heating system 114 provides radiant heating to the wet solids by infrared heating elements within infrared heating system 114. Infrared heating system 114 can include one infrared heating element or a plurality of infrared heating elements. Furthermore, the heating elements can have either the same or different heating outputs from each other and may be electric, gas, or liquid propane. Once the wet solids are processed into dry solids within drum 116, the rotation of drum 116 is reversed to the counter-clockwise direction. In the counter-clockwise direction, drum 116 channels the dry solids toward the front end 154 such that the dry solids are ejected through radial exit openings 174 defined within the front end 154 of drum 116 and pass through the open bottom 106. The exit openings 174 are described further below.

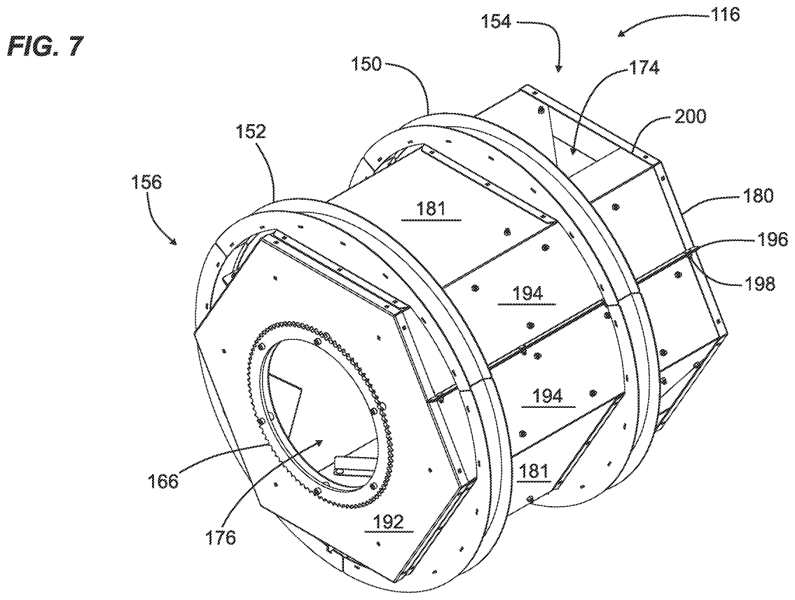

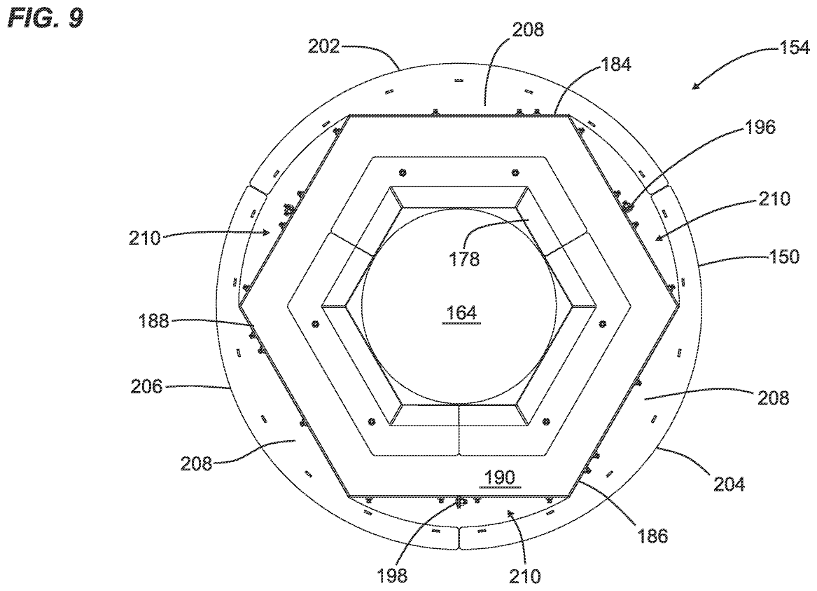

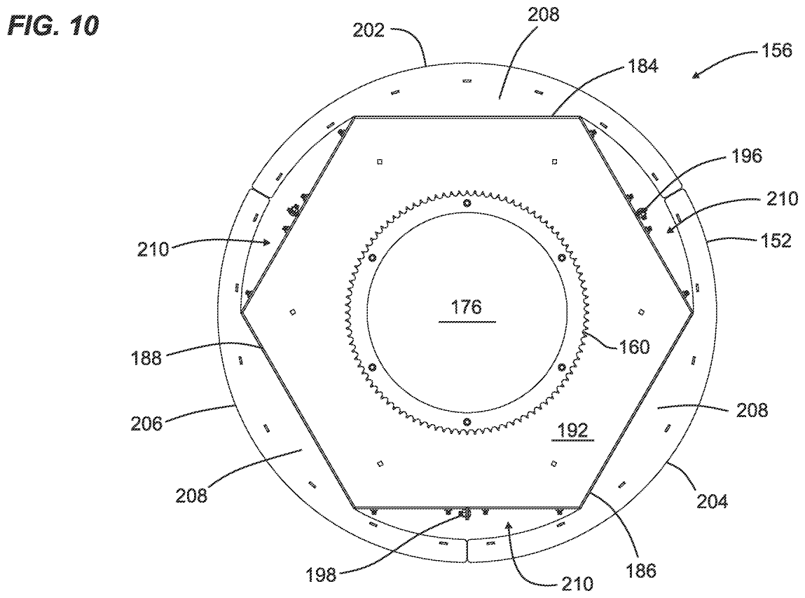

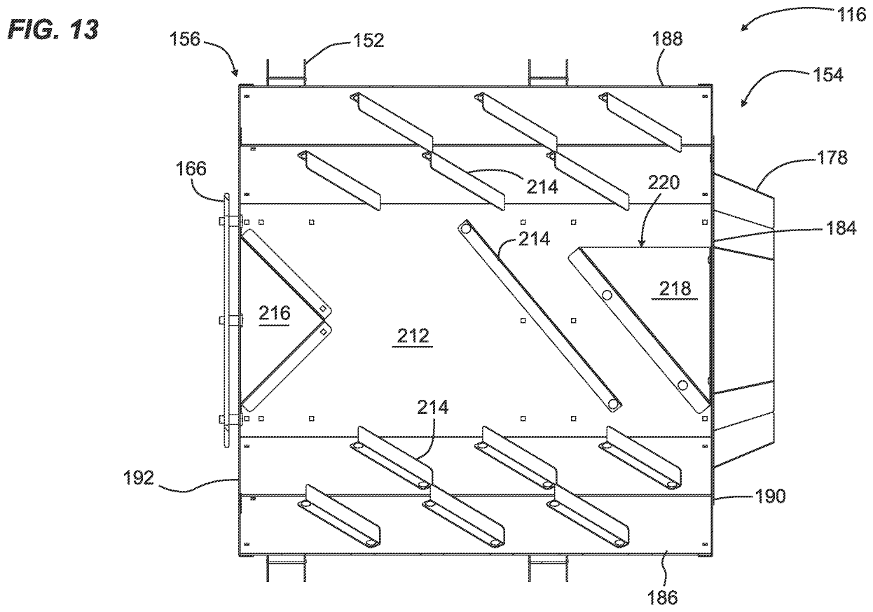

Referring now to FIGS. 7-13, the drum 116 is disclosed in further detail. As described above, drum 116 extends along longitudinal axis 146 having the front end 154 and the back end 156. Back end 156 defines a circular opening 176 such that infrared heating system 114 may extend into drum 116 from frame assembly 112 and is fixed with respect to the rotation of drum 116. Axially adjacent to opening 176, gear 166 is coupled to the back end 156 such that drum 116 is rotatable about longitudinal axis 146 through drive motor 118. The front end 154 also defines opening 164 such that wet solids can be loaded into drum 116 through loading assembly 144. Extending axially outward from front end 154, a flange cover 178 surrounds opening 164 to facilitate retention of the wet solids within drum 116 during loading and processing.

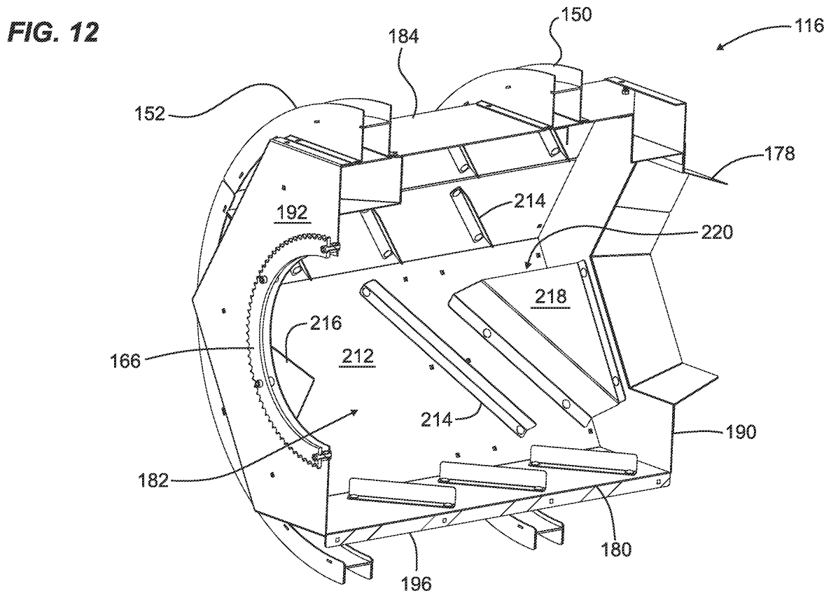

In the example, drum 116 includes a body 180 that has a hexagonal prism shape with six planar sides 181 extending between a hexagonally-shaped front end 154 and back end 156. Body 180 defines an interior cavity 182, best shown in FIG. 12, that receives the wet solids. In alternative examples, body 180 may have any other prismatic polyhedron shape including, but not limited to, triangle, square, pentagon, heptagon, and octagon. As shown, body 180 includes three similarly shaped side members 184, 186, and 188 and two end members 190, 192 that are coupled together and form body 180.

For example, each side member 184, 186, and 188 is approximately one-third of the circumferential perimeter of body 180 and extends longitudinally from front member 190 to back member 192. Each side member 184, 186, and 188 is formed from one planar side 181 and half of both the adjacent planar sides forming a trapezoid shape with two free legs 194. As such, three side members 184, 186, and 188 may be coupled together to form the six hexagonal planar sides 181 of body 180 with three full planar sides 181 and three sides that include two free legs 194. At the free end of each free leg 194, a flange 196 is included to enable each side member 184, 186, and 188 to be coupled together, for example, via a through-bolt and a nut 198. In alternative examples, each side member 184, 186, and 188 may be coupled together via any other method that enables drum 116 to function as described herein.

The front and back members 190, 192 are hexagonally-shaped so as to couple to the side members 184, 186, and 188 at both longitudinal ends 154, 156 and complete the body 180. Around the perimeter of front and back members 190, 192, a flange 200 is included to enable each member 190, 192 to be coupled to the side members 184, 186, and 188, for example, via through-bolt and nut 198, or any other connection method. In alternative examples, body 180 may be formed from any other number of members and configurations, including unitary construction, which enables drum 116 to function as described herein. Drum 116 may be constructed out of sheet metal and as such, by forming body 180 as a hexagonal prism shape with planar sides 181, manufacturing time may be reduced because the sheet metal is not formed into curved sections, which is a time-consuming process. Additionally, the planar sides 181 intersect at an angle to form interior cavity 182, the angled side intersections of drum 116 facilitate increasing solids turn over during rotation of drum 116, as the solids tend to accumulate at the intersection angle and then release to the top layer of solids from the rotational movement.

In the example, each rib 150, 152 is also split into three members 202, 204, and 206 that are approximately one-third of the circumferential perimeter of the rib 150, 152 and coupled to the exterior surface 148 of the full planar sides 181 of each side member 184, 186, and 188. Each rib member 202, 204, and 206 has an arcuate channel 158 defined in the outer perimeter and a radial support 208 that extends from approximately the midpoint of channel 158 to enable the rib member 202, 204, 206 to be coupled to the exterior surface 148 of each respective side member 184, 186, 188. When each rib member 201, 204, 206 is coupled to body 180, the ribs 150, 152 are cylindrical in shape and extend circumferentially around body 180. As each rib 202, 204, 206 is coupled to only the full planar side 181 of body 180, a gap 210 is formed between each rib 150, 152 and free legs 194 enabling the flange 196 connection to extend within the gap 210. Ribs 150, 152 provide rotational support for drum 116 within housing 102 via roller assemblies 160, 162 as discussed above.

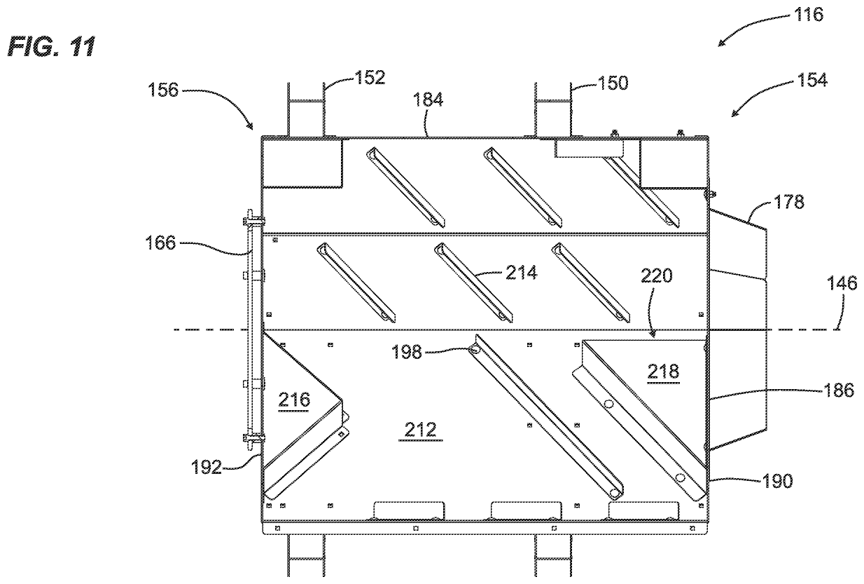

FIGS. 11-13 show cross-sections of drum 116. Body 180 includes an interior surface 212 having a plurality of flights 214 that extend into the interior cavity 182. In the example, flights 214 facilitate channeling wet solids towards the back end 156 of drum 116 and lifting the solids residing at the bottom of the interior cavity 182 towards the lop layer of solids for infrared drying while the drum 116 is rotating clockwise during the drying operation. Flights 214 also facilitate channeling the dried solids towards the front end 154 of the drum 116 while the drum 116 is rotating counter-clockwise during the unload operation. As shown, flights 214 are L-shaped channels that are coupled to body 180 via a through-bolt and nut connection 198 such that flights 214 are replaceable once wear occurs. Flights 214 also extend from the front end 154 to the back end 156 at an angle a relative to the longitudinal axis 146, for example, flights 214 are at an obtuse angle.

In the example, each side member 184, 186, 188 includes a similar configuration of flights 214. As such, on each free leg 194, three parallel flights 214 are coupled to the interior surface 212, and on the planar side 181, a single flight 214 is coupled to the interior surface 212. Additionally, on the full planar side 181 on each side member 184, 186, 188, a back projection 216 is coupled to the interior surface 212 and adjacent to back member 192. Back projection 216 extends within interior cavity 182 and is triangle-shaped to enable the solids channeled toward back end 156 to be pushed back out towards the middle of drum 116 during both clockwise and counter-clockwise operation.

Furthermore, on the full planar side 181 on each side member 185, 186, 188, an exit projection 218 is coupled to the interior surface 212 adjacent front member 190 and extending above exit opening 174. Exit projection 218 is triangle-shaped with an opening 220 defined on the counter-clockwise face of the projection 218 such that during counter-clockwise rotation of drum 116, solids may be channeled through opening 220 and radially pass through exit opening 174. The exit opening 174 is positioned between front end 154 and rib 150 such that during counter-clockwise rotation of drum 116, the dry solids are expelled from the interior cavity 182 and can exit from housing 102 at the open bottom 106 so as not to be retained in the housing 102.

From the examples presented above, one skilled in the art will understand that an infrared drying system can be constructed in many different configurations without departing from the concepts presented in this disclosure. For example, the infrared drying system could have more or fewer interior drum flights; more or fewer infrared heating elements of the same or different capacities; and manual or fully automated process controls. One skilled in the art will understand from the disclosure that many other variations exist as well.

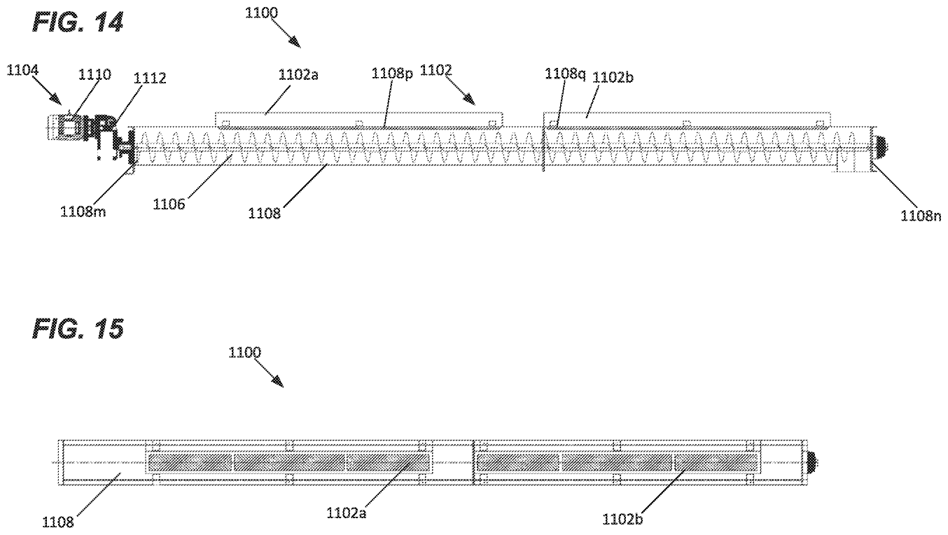

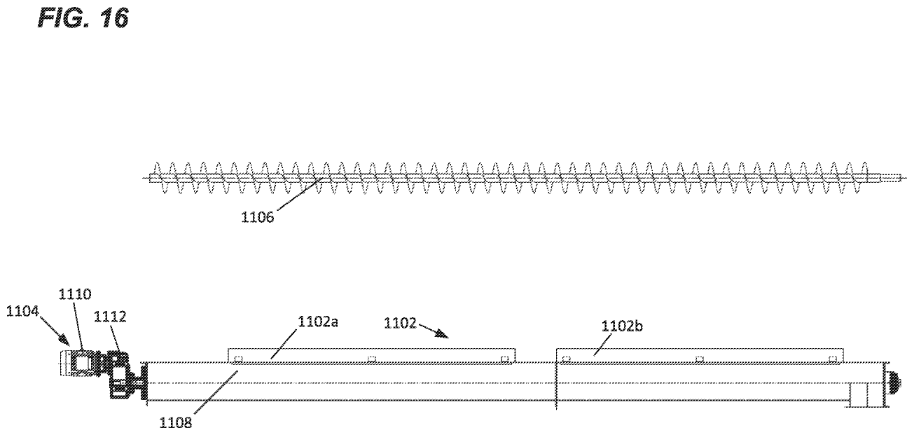

Referring to FIGS. 14-16, a second embodiment of an infrared drying system 1100 is disclosed. Infrared drying system 1100 is used for lowering the moisture content of wet solids to result in dried solids. By use of the term dried solids, it is meant to include solids that have a reduced moisture content as compared to the moisture content of the entering wet solids stream. As most easily seen in FIGS. 14 and 15, infrared drying system 1100 includes a heating system 1102 including a pair of infrared heaters 1102a, 1102b disposed above an auger conveyance system 1104. Examples of suitable infrared heaters 1102 are disclosed in US patent publication 2013/0174438 published on Jul. 11, 2013, the entirety of which is incorporated by reference herein. The infrared heating system 1102 provides radiant heating to wet solids by the infrared heating elements 1102a, 1102b within infrared heating system 1102. Infrared heating system 1102 can include one infrared heating element or more than two infrared heating elements. Furthermore, the heating elements can have either the same or different heating outputs from each other and may be electric, gas, or liquid propane.

The auger conveyance system 1104 is shown as including an auger 1106 disposed within a conveyance tube 1108. The conveyance tube 1108 extends between a first open end 1108m and a second open end 1108n. When the auger 1106 rotates within the tube 1108, material within the tube 1108 will be transported from the first open end 1108m to the second open end 1108n. In the example shown, the auger 1108 is driven by an electric motor 1110 through a power transmission link 1112, such as a gearing or pulley system. Other drive methods may be utilized.

In one aspect, the conveyance tube 1108 includes openings 1108p, 1108q in the sidewall of the tube along its length such that the infrared heaters 1102a, 1102b can be mounted to the tube and apply infrared heating to the material being conveyed within the tube 1108. As shown, the infrared heaters 1102a, 1102b are mounted directly to the tube 1108.

The motor 1110 and power transmission link 1112 can be configured to maintain a predetermined feed rate such that the material being transported through the tube 1108 has a preset travel time between the first and second open ends 1108m, 1108n of the tube to ensure that the material is exposed to the infrared heaters 1102a, 1102b for a predetermined period of time. The heating output of the infrared heaters 1102a, 1102b can also be set to a predetermined output. The auger speed and heater output, and predetermined period of time can be calculated based on a certain material needing to reach a given temperature or level of dryness before it reaches the second end of the tube 1108.

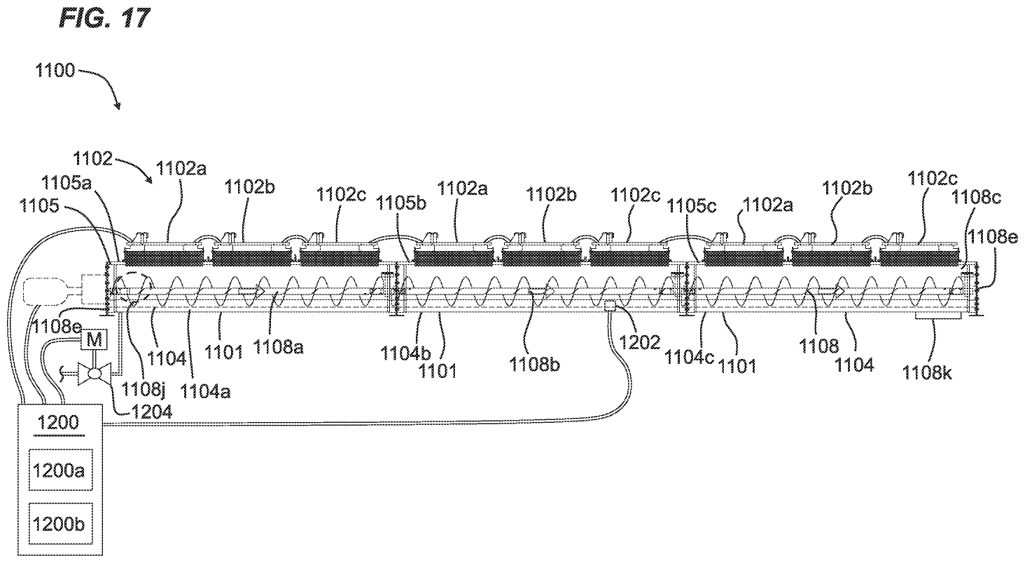

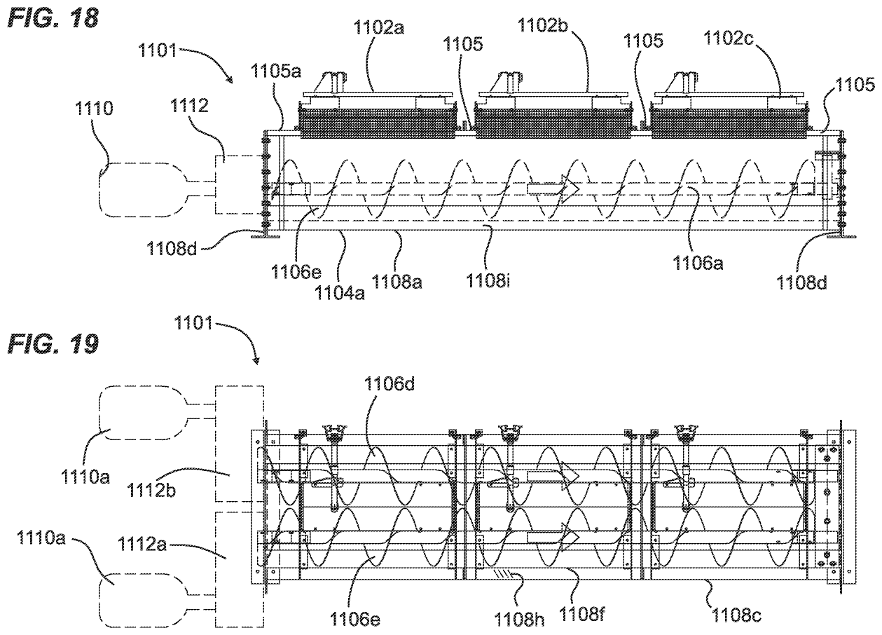

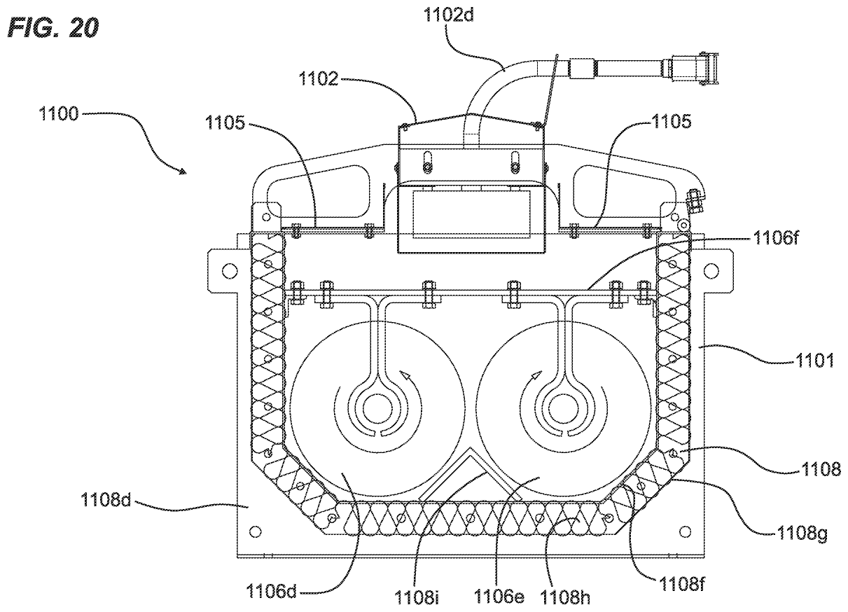

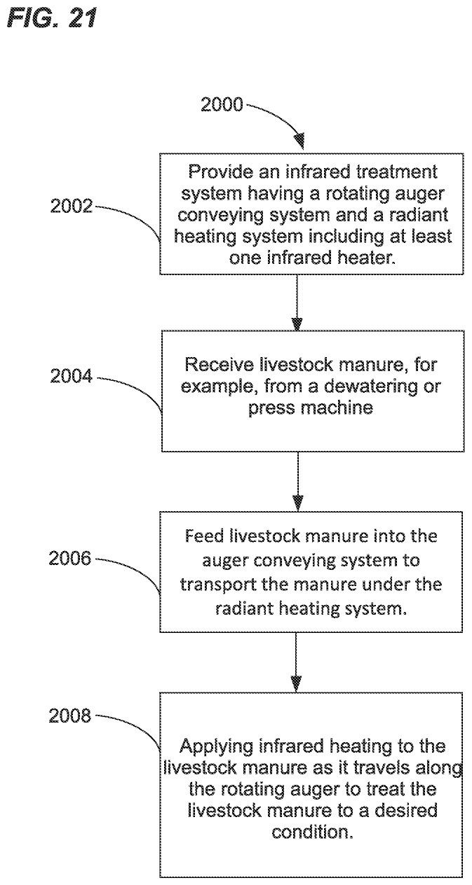

Referring to FIGS. 17-20, a second embodiment of an infrared drying system 1100 is disclosed. Infrared drying system 1100 may also be referred to as an infrared treatment system as, in some applications, pathogens are removed from the wet solids without drying the wet solids beyond a fifty percent moisture content. As such, infrared treatment system 1100 is used to simultaneously lower the moisture content and pathogen levels of wet solids to result in dryer, pathogen free solids. As many aspects of the system 1100 shown in FIGS. 17-20 are similar to those already shown and described for the system 1100 shown in FIGS. 14-16, they need not be repeated here. Therefore, the description for the system 1100 shown at FIGS. 17-20 will be limited to the differences between the embodiments. The primary differences are that the treatment system 1100 shown in FIGS. 17-20 has a modular design that uses a two counter-rotating augers. The treatment system 1100 shown at FIGS. 17-20 also includes an insulated trough design with enhanced manufacturability aspects. As is explained in more detail below, other differences exist as well.

In one aspect of the design, wet solids (e.g., untreated livestock manure) introduced into the treatment system 1100 are heated by infrared heaters 1102a, 1102b, 1102c to achieve 99+ percent pathogen kill while at the same time not making the product too dry and therefore unusable as bedding. Pathogens in bedding have been proven to be a primary driver of mastitis infection in dairy cows. Killing these pathogens reduces mastitis, improves the health of the herd, and raises the value of the milk through reduced SCC counts. At the same time, dairy cows do not like bedding that is more than 50 percent dry (by weight). Bedding that is more than 50 percent dry can blow around in a barn environment, stick to the cow's teats, get in their eyes, and causes general discomfort that translates into lost milk production. Some prior art systems are capable of killing pathogens in wet solids, but teach drying the solids to at or below 5 percent moisture content, and are thus unsuitable for treating wet solids that will be used for bedding material. The disclosed system removes pathogens from wet solids, such as livestock manure with a typical initial moisture content of about 65 to 70 percent by weight, but without drying the wet solids to below a moisture content of 50 percent by weight. The disclosed system 1100 is able to achieve pathogen kill without reducing the moisture content below 50 percent by weight without passing a heated airstream over or across the wet solids, thereby saving on equipment manufacturing and operating costs while improving reliability of the system.

In one aspect, the infrared treatment system 1100 presented at FIGS. 17-20 includes a plurality of modules 1101 attached together to form the treatment system 1100. As described below, each module is provided with an auger section 1106 that can connect with the auger section 1106 of an adjacent module 1101. Each module can also be provided with a heating system 1102 as well. With such a design, the number of modules 1101 can be selected to suit any given application. The individual modules 1101 can also be transported more easily prior to assembly on an installation site. In one example, each module is about 10 feet in length. Additionally, it is possible to install intermediate curved sections between the modules to form an L-shaped or U-shaped system 1100, which is desirable in some installations where an entirely straight system may not fit within the building in which the system 1100 is installed. Although the system 1100 is shown as being modular, the system 1100 could be provided as a continuously built system without individual modular sections.

In one aspect, the infrared treatment system is provided with a heating system 1102 including a plurality of infrared heaters 1102a, 1102b, 1102c disposed above an auger conveyance system 1104. In the example shown, each module 1101 is provided with three infrared heaters 1102a, 1102b, 1102c, giving the heating system 1102 a total of nine infrared heaters. Examples of suitable infrared heaters 1102 are disclosed in US patent application publication 2013/0174438 published on Jul. 11, 2013, the entirety of which is incorporated by reference herein. The infrared heating system 1102 provides radiant heating to wet solids by the infrared heating elements 1102a, 1102b, 1102c within infrared heating system 1102. Infrared heating system 1102 can include one infrared heating element or more than three infrared heating elements. Furthermore, the heating elements can have either the same or different heating outputs from each other and may be electric, gas, or liquid propane. In one example, the infrared heaters 1102 are connected to a power source via power cables 1102d and have electrically powered heating elements utilizing KANTHAL™ wire (e.g. ferritic iron-chromium-aluminum alloy—FeCrAl alloy which can be heated up to over 2,500 degrees F. By providing a radiant heat output at such elevated temperatures, for example, about 2,000 degrees F., in combination with agitating wet solids (e.g., dairy cow manure) with the auger system 1106, pathogens can be quickly killed without having to reduce the moisture content to extremely low levels (e.g., at or below 5 percent by weight). By use of the term about 2,000 degrees F., it is meant to include temperatures between 1,800 degrees and 2,200 degrees.

The infrared treatment system 1100 is also shown as being provided with an auger conveyance system 1104 which is shown as including an auger system 1106 disposed within a conveyance trough, channel, or tube 1108 and covered by a cover 1105. The cover 1105 encloses the open top of the conveyance tube 1108 at locations not already covered by the heating system 1102. As the disclosed system is modular, the conveyance system 1104 is provided with three sections 1104a, 1104b, 1104c with corresponding conveyance tube sections 1108a, 1108b, 1108c, cover sections 1105a, 1105b, 1105c, and auger system sections 1106a, 1106b, 1106c. As shown, each conveyance tube section 1108a, 1108b, 1108c is provided with a flange 1108d at each end such that the sections can be interconnected, such as by bolts. At the ends where no other section is attached, a plate 1108e may be attached to the flange section 1108d to enclose the ends of the conveyance tube 1108. Each conveyance tube section is also provided with an inner wall 1108f and an outer wall 1108g, between which insulation 1108h, such as fiberglass batting insulation, is installed. In the example shown, the inner wall 1108f is formed from stainless steel to increase corrosion resistance. As the heating system 1102 includes heaters operating at a very high temperature (e.g., 2000 degrees F.), the double-wall, insulated construction acts to retain heat within the conveyance tube 1108 and to prevent the outside of the conveyance tube 1108 from reaching excessive temperatures.

In one aspect, the disclosed design incorporates fabrication elements that make it significantly cheaper and more efficient to produce. For example, each of the inner and outer walls 1108f, 1108g are provided with a U-shaped construction with each wall having only four bends. Although more bends can be utilized, the disclosed shape is more readily formable from stainless steel sheet, in comparison to some prior art tubes having as many as twenty bends to form a trough. Some prior auger designs incorporate a rounded trough that the auger sits in. To produce such a trough, the fabrication sometimes does not involve actually “rounding” the trough at all. Instead, to achieve the rounded effect, upwards of twenty separate bends must be done with the material. With long sections of stainless sheet metal, it is very difficult to make all of those bends with precision, and the trough can become warped or lopsided. This can make final fabrication difficult and can cause tolerance variances that put extra wear and tear on the auger itself. As such, the disclosed design, which incorporates shorter 10 foot sections 1108a, 1108b, 1108c and a flat bottom trough that only requires four bends, is advantageous from a manufacturability standpoint.

In one aspect, the conveyance tube is provided with a longitudinal wedge structure 1108i extending the length of each section 1108a, 1108b, 1108c. The wedge structure 1108i is disposed along the bottom section of the inner wall 1108f and resides between two augers 1106d, 1106e (discussed below) to eliminate a dead space where the wet solids could accumulate and to ensure that the treated solids are continually forced to contact the augers 1106d, 1106e. In the example shown, the wedge structure 1108i is separately formed and later attached to the inner wall 1108f. However, the wedge and wall could be integrally formed together in an alternative arrangement.

As mentioned previously, the infrared treatment system 1100 includes an auger system 1106 with modular sections 1106a, 1106b, 1106c. In one aspect, the auger system 1106 includes a first auger 1106d and a second auger 1106e, both of which are supported by a bearing assembly including a bearing hanger plate 1106f. In the embodiment shown, the bearing hanger plate 1106f is supported by conveyance tube 1108. In one aspect, the augers 1106e, 1106d include spiral flights and are rotated in a counter-rotating fashion to transfer the treated solids from an inlet end 1108j to an outlet end 1108k of the conveyance tube 1108. The combination of utilizing a dual-auger system in conjunction with high temperature infrared heaters advantageously enables the treated solids to be thoroughly exposed to the heaters to quickly kill pathogens while quickly conveying

In one aspect, the auger system 1106 is driven by an electric drive system 1110 through a power transmission link 1112, such as a gearing or pulley system. In the particular example shown, first and second motors 1110a, 1110b drive first and second power transmission links 1112a, 1112b such that the augers 1106d, 1106e are separately powered. Other drive methods may be utilized. For example, a single motor could power a single transmission link operably connected to each auger 1106d, 1106e.

The motor 1110 and power transmission link 1112 can be configured to maintain a predetermined feed rate such that the material being transported through the tube 1108 has a preset travel time between the inlet and outlet ends 1108j, 1108k of the tube to ensure that the material is exposed to the infrared heaters 1102a, 1102b, 1102c for a predetermined period of time. The heating output of the infrared heaters 1102a, 1102b, 1102c can also be set to a predetermined output. The auger speed and heater output, and predetermined period of time can be calculated based on a certain material needing to kill pathogens at a desired moisture content (e.g., at or above 50 percent moisture content) before it reaches the second end of the tube 1108.

Referring to FIG. 17, an electronic controller 1200 is schematically shown as including a processor 1200a and a non-transient storage medium or memory 1200b, such as RAM, a flash drive, or a hard drive. Memory 1200B is for storing executable code, the operating parameters, and potential inputs from an operator interface, while processor 1200a is for executing the code. Electronic controller 1200 is connected to a number of inputs and outputs that may be used for controlling the infrared treatment system 1100. For example, the electronic controller 1200 includes outputs for controlling the motors 1110 and infrared heaters 1102 and inputs 1202 for monitoring the temperature inside the conveyance tube 1108.

In one exemplary process 2000, that can be implemented by the controller 1200 as shown in FIG. 21, an infrared drying system is provide in a step 2002. In steps 2004 and 2006, livestock manure is received from a source, such as a dewatering machine or a press machine, and then fed into the above described auger conveyance system 1104 such that the livestock manure is conveyed along the tube 1108 and under the radiant heaters 1102a, 1102b, 1102c. In a step 2008, infrared heating is applied to the livestock manure as it travels through the tube 1108 such that the livestock manure achieves desired condition. In one example, the desired condition is a solids temperature of between 176 and 280 degrees F. to produce dried, sterilized livestock bedding. In one example, the desired condition is a solids condition with 99.9 percent pathogen kill and a moisture content of about 50 percent by weight. By use of the term about 50 percent, it is meant to include percentages between 40 and 60 percent. In some examples, the desired condition is a solids condition with 99.9 percent pathogen kill and a moisture content above 50 percent by weight. In one example, the auger speed is set to a predetermined speed such that the livestock feed is exposed to the heaters 1102a, 1102b, 1102c for a predetermined length of time. In one example, the entering livestock manure has a moisture content of about 65 to 70 percent by weight and the motor/auger speed is set such that the livestock manure will travel the entire length (e.g., 32 feet) of the conveyance tube 1108 in about 12 minutes. This time period is generally equal to the exposure or dwell time that the livestock manure is exposed to the heaters 1102a, 1102b, 1102c. In some examples, the transport or dwell time is about 10 minutes. At exit, the livestock manure has been dried to have a reduced moisture content of about 50 percent by weight with a 99.9 percent pathogen kill. With the disclosed infrared treatment system 1100 presented at FIGS. 17 to 20, about 1.5 tons of livestock manure can be continuously treated in this fashion. In some examples, the cow manure is heated between 176 degrees F. and 280 degrees F. as it moves toward the opposite end of the auger.

In another control process, a water injection valve 1204 can be opened if the temperature monitored at a temperature sensor (e.g., sensor 1202) exceeds a threshold valve. In one example, the temperature sensor can be positioned to measure the temperature of the treated solids. Such a process would be implemented where conditions for a fire within the conveyance tube 1108 may exist. As the infrared heaters operate at a relatively high temperature, the risk for the solids to burn increases. The water injection valve 1204 is shown as being connected to a water source and to the conveyance tube 1108. As such, when the water injection valve 1204 is opened, water floods the conveyance tube 1108. Such a control process can also include shutting down the infrared heaters 1102a, 1102b, 1102c and generating an alarm (e.g., an audible alarm, a visible alarm, an alarm signal sent to a user interface, and/or an alarm sent to a remote location) when the temperature sensor reaches a predetermined value (e.g., 300 degrees F. to 400 degrees F.).

Various modifications and alterations of this disclosure will become apparent to those skilled in the art without departing from the scope and spirit of this disclosure, and it should be understood that the scope of this disclosure is not to be unduly limited to the illustrative embodiments set forth herein.

1. A rotatable drum for an infrared drying system comprising:
a. a body comprising:
i. a first end defining an entry opening;
ii. an opposite second end;
iii. a plurality of planar sides extending between the first end and the second end, at least one of the plurality of planar sides defining an exit opening adjacent to the first end; and
iv. an interior cavity defined by the first and second ends and the plurality of planar sides, the interior cavity configured to receive solids at the entry opening and discharge solids at the exit opening; and
b. at least one flight extending from an interior surface of the body into the interior cavity, the at least one flight configured to channel solids from the entry opening to a back end when the drum is rotating clockwise and to channel solids from the second end to the exit opening when the drum is rotating counter-clockwise.

2. The rotatable drum of claim 1, wherein the body comprises six planar sides.

3. The rotatable drum of claim 2, wherein the six planar sides are formed from three members.

4. The rotatable drum of claim 1, further comprising at least one circumferential rail coupled to an exterior surface of the body, the at least one circumferential rail configured to support the body within a housing of the infrared drying system.

5. The rotatable drum of claim 1, wherein the at least one flight includes two parallel flights.

6. The rotatable drum of claim 1, wherein the body defines a longitudinal axis and the at least one flight extends in a direction from the first end to the second end at an obtuse angle from the longitudinal axis.

7. The rotatable drum of claim 1, further comprising an exit projection extending from the interior surface of the body into the interior cavity above the exit opening, the exit projection open at one end such that when the drum is rotating counter-clockwise, solids are channeled to the exit opening.

8. The rotatable drum of claim 1, further comprising a back projection extending from the interior surface of the body into the interior cavity adjacent to the back end, the back projection configured to channel solids from the back end towards the entry opening during rotation of the drum.

9. An infrared treatment system for killing pathogens in a waste stream, the infrared treatment system comprising:
a. a conveyance tube extending from an inlet end to an outlet end, the conveyance tube having a double wall construction with insulation disposed between an inner wall and an outer wall, the conveyance tube having an open top side;
b. a plurality of infrared heaters mounted to the conveyance tube and at least partially covering the open top side such that heating elements of the infrared heaters are exposed to an interior of the conveyance tube;
c. a plurality of covers mounted to the conveyance tube and covering portions of the open top side of the conveyance tube not covered by the plurality of infrared heaters;
d. a first auger and a second auger disposed within the conveyance tube, the first and second augers being driven by a drive system in a counter-rotating configuration such that material fed into the conveyance tube inlet end is transported to the outlet end of the conveyance tube, and is exposed to the heating elements of the infrared heaters as the material is transported between the inlet end and the outlet end;
e. a water injection system, the water injection system including a valve operable to flood the conveyance tube at a predetermined sensor temperature; and
f. a control system configured to, when the predetermined sensor temperature is exceeded: open the valve to flood the conveyance tube, deactivate the heating elements, and generate an alarm.

10. The infrared treatment system of claim 9, wherein each the plurality of infrared heaters includes a heating element operating at 2,000 degrees F.

11. The infrared treatment system of claim 9, wherein the inner wall is formed from stainless steel.

12. The infrared treatment system of claim 9, wherein the infrared treatment system is constructed from multiple modules, wherein at least two of the modules include an infrared heater and a section of the first and second augers.

13. The infrared treatment system of claim 12, wherein the modules each have a length of about 10 feet.

14. The infrared treatment system of claim 9, wherein the heating elements include ferritic iron-chromium-aluminum alloy wire heating elements.

15. The infrared treatment system of claim 9, wherein the predetermined sensor temperature is a temperature of solids conveyed within the conveyance tube.

16. The infrared treatment system of claim 9, wherein the inner wall includes four straight segments separated by four bend lines.

17. The infrared treatment system of claim 9, further comprising a wedge structure extending between and parallel to the first and second augers.

18. A method for treating livestock manure comprising:
a. receiving livestock manure at an inlet end of a conveyance tube;
b. transporting the livestock manure to an outlet end of the conveyance tube;
c. exposing the livestock manure to heating elements of an infrared heater while the livestock manure is being transported from the inlet end to the outlet end, wherein the heating elements are operating at a temperature of about 2,000 degrees F.; and
d. setting at least one of an auger speed and a heating element output such that the livestock manure is treated to eliminate 99 percent or more of the pathogen content of the livestock manure and to have a reduced moisture content of about 50 percent.

19. The method of claim 18, wherein the method includes injecting water into the conveyance tube and deactivating the infrared heaters when a predetermined temperature threshold is exceeded within the conveyance tube.

20. The method of claim 18, wherein the livestock manure has an initial moisture content of about 65 to 70 percent by weight and a final moisture content of about 20 percent less than the initial moisture content.
